SEQUENCE LISTING (2 sequences)

SEQ ID NO: 1 - DNA, 741 bp, Artificial Sequence
  Synthesized
atgggcagcagccatcatcatcatcatcacagcagcggcctggtgccgcgcggcagccatatgctcgaggatcccatgaagtttagaagtataatccttgcagggctccttgttaccctgcttgcaccatcagttaaagctcagtttaccgttcctgatttgccttatgcctttgatgcccttgagcccgcaattgataaggagacgatgcagatccatcacgataagcatcatgctgcatatgtaaaaaatcttaacgacgcggttaagggaactgcccaagaaaagcaaacccttgctcaaatcctggcagcggtgtctaaagcttcccctgcagtacgaaataatgctggcggacattataaccacagcctcttctggcagatcatggcgccgaaagcacaggctccttctgccgcatttctaaaggtgatcgatgcacagtttggttctcttgataagtttaaagccgcctttgctgactccgccgccaagcgctttggttcaggatgggcctggctgattgttcagaaaggtaagttgaagatcaccaccactcctaatcaggacaatccgctgatggatgttgtaaaggagaagggaacgccgatcctggccctcgacgtttgggaacacgcctactatctgaagtatcagaacaagcggcctgactatatctccgcatggtggacggtcgtaaactggccggaggttgagagacggtataaccagataaaatag

SEQ ID NO: 2 - protein, 246 aa, Artificial Sequence
  Synthesized
MGSSHHHHHHSSGLVPRGSHMLEDPMKFRSIILAGLLVTLLAPSVKAQFTVPDLPYAFDALEPAIDKETMQIHHDKHHAAYVKNLNDAVKGTAQEKQTLAQILAAVSKASPAVRNNAGGHYNHSLFWQIMAPKAQAPSAAFLKVIDAQFGSLDKFKAAFADSAAKRFGSGWAWLIVQKGKLKITTTPNQDNPLMDVVKEKGTPILALDVWEHAYYLKYQNKRPDYISAWWTVVNWPEVERRYNQIK

(19) United States
(12) Patent Application Publication
(10) Pub. No.: US 2020/0149016 A1
(43) Pub. Date: May 14, 2020

(54) SUPEROXIDE DISMUTASE GENE AND ITS CODING PROTEIN

(71) Applicant: FUZHOU UNIVERSITY, FUZHOU (CN)

(72) Inventor: JUN LIN, FUZHOU (CN)

(73) Assignee: FUZHOU UNIVERSITY

(21) Appl. No.: 16/749,381

(22) Filed: Jan. 22, 2020

Related U.S. Application Data
(63) Continuation of application No. PCT/CN2018/093736, filed on Jun. 29, 2018.

(30) Foreign Application Priority Data
Jul. 28, 2017 (CN) 201710626682.0

(51) Int. Cl. C12N 9/02

(52) U.S. Cl. CPC C12N 9/0089; C12Y 115/01001

(57) ABSTRACT

The present invention discloses a superoxide dismutase gene and a protein encoded thereby, the DNA sequence of the superoxide dismutase gene is shown in SEQ ID NO. 1, and the encoded protein sequence of the superoxide dismutase gene is shown in SEQ ID NO 2. The superoxide dismutase encoded by the SOD gene of the invention has good heat resistance and freeze-thaw resistance, and can be widely applied in the fields of cosmetics, food, medicine, environmental protection and the like.

### CROSS-REFERENCE TO RELATED APPLICATIONS

This application is a continuation of International Patent Application No. PCT/CN2018/093736 with a filing date of Jun. 29, 2018, designating the United States, now pending, and further claims priority to Chinese Patent Application No. 201710626682.0 with a filing date of Jul. 28, 2017. The content of the aforementioned applications, including any intervening amendments thereto, are incorporated herein by reference.

### BACKGROUND OF THE INVENTION

### 1. Field of the Invention

The invention relates to biological field, and specifically to a superoxide dismutase gene and its coding protein.

### 2. Description of Related Art

Superoxide Dismutase, alias liver protein or SOD, is an important antioxidant enzyme in the body and widely present in various organisms such as animals, plants and microorganisms. SOD has special physiological activity and is the primary substance for scavenging oxygen free radicals in living organisms. The level of SOD in the body is an indicator of aging and death. It has been confirmed that there are more than 60 diseases caused by oxygen free radicals. It can restore the damage caused by oxygen free radicals, and repair damaged cells in time. Due to the pressure of modern life, environmental pollution, various radiation and excessive movement will cause a large number of oxygen free radicals. Therefore, the role of SOD in the biological antioxidant field is becoming more and more important.

Superoxide dismutase can be classified into three types according to different metal cofactor contained therein. The first type is SOD containing copper and zinc metal cofactor, called Cu.Zn-SOD. It is the most common SOD enzyme, which is green and mainly exists in the cytoplasm; the second is manganese metal cofactor containing, this SOD called Mn-SOD, is purple, present in the prokaryotic cells and mitochondria of eukaryotic cells; the third is iron metal cofactor containing, called Fe-SOD, which is yellowish brown and exists in prokaryotic cells.

SOD has many functions of anti-aging, immune regulation, regulation of blood lipids, anti-radiation, skin care and so on, and has important applications in the fields of cosmetics, food, medicine, and environmental protection. However, defects in heat resistance and freeze-thaw resistance limit the application of the prior SOD.

### SUMMARY OF THE INVENTION

The object in the art is to overcome the deficiencies of the prior art and to provide a superoxide dismutase gene and a protein encoded thereby, the superoxide dismutase encoded by the gene having good heat resistance and freeze-thaw resistance.

The superoxide dismutase gene (SOD gene) provided in this invention has a DNA sequence as shown in SEQ ID NO.1

The present invention also provides a protein encoded by the above superoxide dismutase gene, the protein sequence of which is shown in SEQ ID NO.2

The SOD gene of the present invention is origin from a natural environment water sample collected from Minhou County, Fuzhou City, Fujian Province. The metagenomic DNA of it was extracted, and the extracted DNA is directly used in PCR amplification.

The superoxide dismutase encoded by the SOD gene of this invention has good heat resistance and freeze-thaw resistance, and can be widely applied in the fields of cosmetics, food, medicine, environmental protection and the like.

### BRIEF DESCRIPTION OF THE DRAWINGS

FIG. 1 is an electrophoresis gel picture of the PCR product in the first step of Example 1.

FIG. 2 is an electrophoresis gel picture of the step 6 of Example 1. The upper row of 1 to 6 wells from the left: DNA marker, sample 6 to 10; the lower row of 1 to 7 wells from the left: DNA marker, negative control, and sample 1 to 5.

FIG. 3 is a graph showing the result of SOD activity measurement in Example 2.

### DESCRIPTION OF THE EMBODIMENTS

The following examples are provided for a better understanding of the present invention; however, the present invention is not limited thereto.

The DNA sequence of the superoxide dismutase gene of the present invention is shown in SEQ ID NO.1

The encoded protein sequence of the above superoxide dismutase gene is shown in SEQ ID NO.2

The source of the SOD gene of the present invention is from a natural environment water sample collected from Minhou, Fuzhou, Fujian Province. Using a 0.22 μm membrane filter to retain various organisms therein, the metagenomic DNA of it was extracted with a kit (Mobio PowerDNA Isolation Kit, 14900-50-NF, USA), and the extracted DNA is directly used in PCR amplification as the template DNA.

### Example 1

Construction of the protein expression vector for SOD gene by molecular cloning technology includes the following steps:

Step1, PCR system (50 μL): template DNA 1 μL, Sodinfupet15F primer (10 μM) 2 μL, Sodinfupet15R primer (10 μM) 2 μL, 2×KOD PCR mix (ABM, Canada) 25 μL, and ddH₂O up to 50 μL.

Sodinfupet15F primer sequence:
GTTAGCAGCCGGATCCTATTTTATCTGGTTATACCGTCTCTCAACCTCC
Sodinfupet15R primer sequence:
ATATGCTCGAGGATCccATGAAGTTTAGAAGTATAATCCTTGCAGGGC

PCR program: 94° C. for 10 min, 30 cycles of (94° C. for 30 s, 50° C. for 30 s, and 72° C. for 45 s), and 72° C. for 5 min.

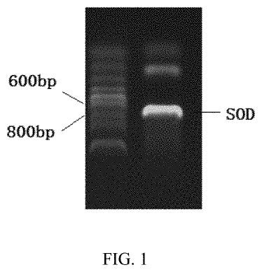

The PCR product was confirmed by agarose gel electrophoresis. The result was shown in FIG. 1. The first lane from the left of FIG. 1 is the marker, the second lane is the PCR product, and the destination band position is shown in FIG. 1.

The PCR product obtained in this example was sequenced using the Sanger method by the ABI 3730x1 sequencer, and the sequencing primers were Sodinfupet15F and Sodinfupet15R. The sequencing experiment was performed by the Guangzhou Yingjun Biotech Co., Ltd. The full length DNA sequence of the superoxide dismutase gene in the present invention was obtained from the Sanger sequencing result, as shown in SEQ ID NO.1.

Step2, The gel purification of the target band of the PCR product from step 1 was carried out with an Omega Gel Extraction Kit (Cat No. D2500-01). The gel-purified PCR product (SOD enzyme gene) serves as a substrate for subsequent in fusion ligation reactions for the construction of the protein expression vector.

Step3, Preparation of the linearized expression plasmid. The pET15b plasmid was digested at 37° C. for 30 min in the follow system: pET15b plasmid (108.8 ng/μL) 10 μL, 10× FastDigest buffer 2 μL, FastDigest BamHI 1 μL, ddH₂O 7 μL. The digested product was identified by agarose gel electrophoresis, and the band of the digested product was purified by the above omega kit. The purified product's concentration was 32 ng/μl. This prepared linearized plasmid was used for the subsequent In fusion ligation.

Step4, In-Fusion Ligation. The purified PCR product is ligated to the linearized pET15b plasmid to construct the expression vector.

The ligation system: 5× In-fusion HD Enzyme Premix (Clontech, USA) 2 μL, linearized pET15b plasmid (32 ng/μL) 1.6 μL (in total about Song), gel-purified SOD gene (50 ng/μL) 1 μL, ddH₂O

The total volume of ligation system is 10 μL.

Incubate at 50° C. for 15 min and then place on ice.

Step5 Transformation.

Take 10 μl of the In fusion ligation product, add to E. coli stb13 competent cells, mix, ice bath for 30 min, heat shock at 42° C. for 45 s, ice bath for 2 min, spread on LB plate (with ampicillin resistance). The plates were incubated for 16 hours in a 37° C. incubator to form single colonies on the plates.

Step6 Verification of Colonies

10 single colonies were picked (numbers 1 to 10) to 10 tubes which containing 10 μl of sterile water each, mixed by pipetting, and 1 μl bacterial suspension of each tube was used as the template for colony PCR to verify whether the target gene was ligated to the plasmid.

The system of colony PCR (Samples):

T7 universal forward sequencing primer 0.4 μl, T7 universal reverse sequencing primer 0.4 μl, Bacterial suspension 1 μl, 2×Taq mix (Cwbiotech, CHINA) 5 μl, ddH₂O 3.2 μL.

The system of colony PCR (Negative Control):

T7 universal forward sequencing primer 0.4 μl, T7 universal reverse sequencing primer 0.4 μl, pET15b plasmid 1 μl, 2×Taq mix (Cwbiotech, CHINA) 5 μl, ddH₂O 3.2 μL.

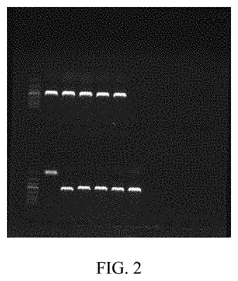

PCR program: 94° C. for 10 min, 30 cycles of (94° C. for 30 s, 50° C. for 30 s, and 72° C. for 60 s), and 72° C. for 5 min. The PCR product was confirmed by agarose gel electrophoresis which was shown in FIG. 2.

Step7, Sequencing verification and construction of protein expression strain.

The sample No. 5 in FIG. 2 was inoculated to liquid LB medium with ampicillin, with cultured overnight, it was sent to Guangzhou Yingjun Biotech Co., Ltd. for sanger sequencing verification with T7 forward and reverse universal sequencing primers. The result showed that the protein expression vector was correct. Then, the plasmid DNA of this sample was extracted and transformed to E. coli ER2566 cells, cultured overnight.

### Example 2

Protein Expression

1. Induced Expression

1) 300 μL of the overnight cultured bacteria in the step 7 of Example 1 was added to 30 mL LB with ampicillin, cultured at 37° C., 200 rpm.

2) The OD value was measured after about 2 hours. When the OD value reached 0.5 (0.3 to 0.5), IPTG was added to a final concentration of 0.1 mM, and then cultured at 20° C., 200 rpm for 12 hours.

2, enzymatic lysis of cells (30 mL bacterial suspension)

1) Collect the cells by centrifugation at 4000 g for 10 min, remove the supernatant, wash once with ddH₂O (resuspend the precipitate in ddH₂O and centrifuge to remove the supernatant).

2) The precipitate corresponding to each 30 mL of bacteria culture is resuspended in 1.2 mL cell lysis buffer (pH 8.5), and the buffer needs to ensure the addition of PMSF.

3) Add lysozyme powder to a final concentration of 1 mg/mL, mix and place at ice bath for 30 min.

4) Transfer the centrifuge tube to the shaker, screw the lid, tilt at 45 degrees, 230 rpm, 25° C., shake for 10 min.

5) Add Triton X-100 12 μL (final concentration 1%), DNase 0.5 μL and RNase 1 μl, (final concentration 5 μg/mL) to the tube, then placed it on a shaker at 230 rpm, 25° C., and shaken for 15 min.

6) Centrifugation at 12000 g for 15 min at 4° C., the supernatant is a soluble protein component. The supernatant was used for subsequent experiments.

The sequence of SOD protein expressed in the supernatant is shown in SEQ ID NO.2

3, SOD Activity Detection

The SOD activity detection kit (WST-8 method) was purchased from the Beyotime Co., Ltd., and the supernatant was directly tested for SOD activity.

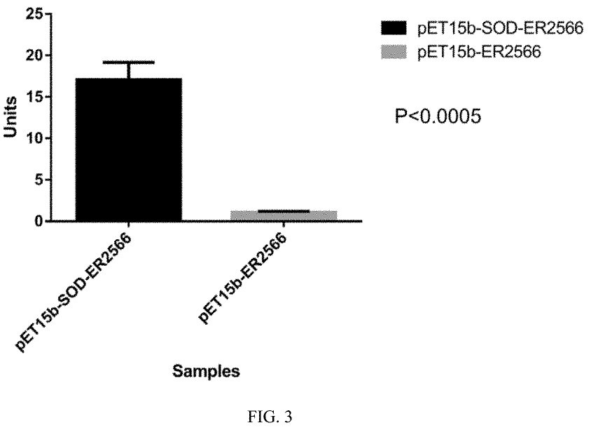

The test results are shown in FIG. 3:

The pET15b-SOD-ER2566 carrying the SOD gene of the present invention has a SOD activity of at least 15 units in the lysate supernatant. The pET15b-ER2566 (negative control) without the SOD gene of the present invention had an activity in the lysate supernatant less than 2.5 Units.

1. Thermal Stability Test

The lysate supernatant of pET15b-SOD-ER2566 carrying the SOD gene of the present invention was incubated at 80° C. for 10 minutes, and the SOD activity was determined, and it was found to retain 90% of the activity, indicating that the SOD enzyme of the present invention has a good thermal stability.

2, Anti-Freezing and Melting Ability

The lysate supernatant of pET15b-SOD-ER2566 carrying the SOD gene of the present invention was placed in an ultra-low temperature refrigerator at −86° C., frozen for 12 hours, taken out, thawed at room temperature (25° C.), and the activity of SOD was measured, and it was found 95% activity remained, indicating that the SOD enzyme of the present invention has good freeze-thaw resistance.

### Example 3

Comparative Test

The SOD enzyme gene under the accession number of SDL36756.1 in GenBank was synthesized by the General Biosystems company, and the gene was cloned into an expression vector according to the methods of Example 1 and Example 2, and the expression of the target protein was induced.

1. Thermal Stability Test

The lysate supernatant of strain which expressing this recombinant protein was incubated at 80° C. for 10 minutes, and the activity of SOD was measured, and it was found to retain 60% of activity compared with the control group which was not treated with high temperature.

2, Anti-Freezing and Melting Ability

The lysate supernatant of strain which expressing this recombinant protein was placed in an ultra-low temperature refrigerator at −86° C., frozen for 12 hours, taken out, and thawed at room temperature (25° C.), and the activity of SOD was measured again, and the control group was treated with no freeze-thaw treatment. In comparison, it was found to retain 75% activity.

According to the heat stability test and the freeze-thaw resistance test of Examples 2 and 3, the superoxide dismutase encoded by the SOD gene of the present invention has good heat resistance and freezing-thawing resistance as compared with the conventional SOD enzyme, broadening its application in cosmetics, food, medicine, environmental protection and other fields.

I claim:

1. A superoxide dismutase gene characterized in that the DNA sequence of the gene is as shown in SEQ ID NO.1

2. The protein encoded by the superoxide dismutase gene according to claim 1, wherein the protein sequence is as shown in SEQ ID NO.2
